(19) United States
(12) Patent Application Publication
(10) Pub. No.: US 2025/0334574 A1
(43) Pub. Date: Oct. 30, 2025

(54) Autoantibody Biomarkers of Ro/SS-A Antibody Negative Sjogren's Syndrome/Sjogren's Disease

(71) Applicants: Oklahoma Medical Research Foundation, Oklahoma City, OK (US); The Board of Regents of the University of Oklahoma, Norman, OK (US)

(72) Inventors: A. Darise Farris, Edmond, TX (US); Sherri Longobardi, Chicago, IL (US)

(21) Appl. No.: 18/875,605

(22) PCT Filed: May 11, 2023
(86) PCT No.: PCT/US2023/066862

§ 371 Date: Dec. 16, 2024

Related U.S. Application Data
(60) Provisional application No. 63/354,875, filed on Jun. 23, 2022.

(51) Int. Cl. G01N 33/564; G01N 33/68

(52) U.S. Cl. CPC G01N 33/564; G01N 33/6854; G01N 2333/4703; G01N 2800/101; G01N 2800/52

(57) ABSTRACT

The present invention includes a method and kit for method for detecting anti-Ro antibody negative Sjögren's syndrome/Sjögren's disease without performing a lip biopsy comprising: obtaining a biological sample from a patient suspected of having an anti-Ro antibody negative Sjögren's syndrome/Sjögren's disease; and detecting if the biological sample has autoantibodies to at least one of: Geminin DNA Replication Inhibitor (GMNN), Kelch Domain Containing 8 A (KLHDC8A), Microtubule Associated Protein RP/EB Family Member 1 (MAPRE1), Nucleoporin 50 (NUP50), or SKI Like Proto-Oncogene (SKIL).

### CROSS-REFERENCE TO RELATED APPLICATIONS

This application claims priority to U.S. Provisional Application Ser. No. 63/354,875, filed Jun. 23, 2022, the entire contents of which are incorporated herein by reference.

### STATEMENT OF FEDERALLY FUNDED RESEARCH

This invention was made with government support under 1R01AR074310 and T32AI007633, awarded by the National Institutes of Health. The government has certain rights in the invention.

### TECHNICAL FIELD OF THE INVENTION

The present invention relates in general to the field of Sjögren's Syndrome (also known as Sjögren's disease), and more particularly to autoantibody biomarkers of Ro/SS-A antibody negative Sjögren's syndrome/Sjögren's disease.

### BACKGROUND OF THE INVENTION

Without limiting the scope of the invention, its background is described in connection with Sjögren's Syndrome.

Sjögren's syndrome (SS)/Sjögren's disease (SjD) is a rheumatic autoimmune disease selectively targeting salivary and lacrimal glands, leading to painful dry mouth and eyes, oral infections, severe dental caries/tooth loss, fatigue, arthritis, nervous system involvement and malignant B cell lymphoma. Current internationally accepted disease classification criteria rely on either the presence of anti-Ro antibodies (these may target either the Ro60 antigen, Ro52 antigen or both) or the presence of focal lymphocytic infiltrates in a minor salivary gland lip biopsy for diagnosis (1, 2).

Among 475 individuals attending the Oklahoma Sjögren's Research clinic and meeting classification criteria for primary SS, 38% lacked antibodies to Ro antigen. These individuals met classification criteria for SS due to a focus score ≥1 on examination of minor salivary gland lip biopsy. The antigen(s) driving the aberrant immune response in these individuals is unknown.

Novel methods are needed to identify Ro/SS-A antibody-negative Sjögren's syndrome/Sjögren's disease patients.

### SUMMARY OF THE INVENTION

As embodied and broadly described herein, an aspect of the present disclosure relates to a method for detecting anti-Ro antibody negative Sjögren's syndrome (SS)/Sjögren's disease (SjD) without performing a lip biopsy comprising: obtaining a biological sample from a patient suspected of having an anti-Ro antibody negative SS/SjD; and detecting if the biological sample has autoantibodies to at least one of: Geminin DNA Replication Inhibitor (GMNN), Kelch Domain Containing 8A (KLHDC8A), Microtubule Associated Protein RP/EB Family Member 1 (MAPRE1), Nucleoporin 50 (NUP50), or SKI Like Proto-Oncogene (SKIL). In one aspect, the method further comprises the steps of detecting if the biological sample has autoantibodies to at least one of SSB or SKIL, and determining that the patient has Sjögren's syndrome/Sjögren's disease without performing a lip biopsy. In another aspect, the method further comprises the step of detecting if the biological sample has autoantibodies to at least one additional biomarkers selected from ADP ribosylation factor GTPase activating protein 1 (ARFGAP1), Chromosome 9 Open Reading Frame 78 (C9orf78), Chromobox 3 (CBX3), Damage Specific DNA Binding Protein 1 (DDB1), GRB2 Associated Binding Protein 1 (GAB1), Heterogeneous Nuclear Ribonucleoprotein A/B (HNRNPAB), ISG15 Ubiquitin Like Modifier (ISG15), Multiple Coagulation Factor Deficiency 2 (MCFD2), NFU1 iron-sulfur cluster scaffold (NFU1), Pleckstrin Homology Domain Containing A4 (PLEKHA4), PML Nuclear Body Scaffold (PML), RNA Polymerase III Subunit H (POLR3H), POU Class 6 Homeobox 1 (POU6F1), RCAN Family Member 3 (RCAN3), SEC23 Interacting Protein (SEC23IP), SRY-box Transcription Factor 5 (SOX5), Small RNA Binding Exonuclease Protection Factor La (SSB), T-complex 10-like 3, pseudogene (TCP10L3), TPD52 Like 1 (TPD52L1), Triosephosphatase 1 (TPI1), Tripartite Motif Containing 21 (TRIM21), Ro60, Y RNA Binding Protein (TROVE2), Zinc Finger And BTB Domain Containing 46 (ZBTB46), and Zinc Finger Protein 655 (ZNF655). In another aspect, the autoantibodies are detected using an assay selected from at least one of: multiplex bead-based assay, capillary Western Blot, ELISA, flow cytometry, fluorimetry, microscopy, immunofluorescence, radioimmunoassay, immunoenzymatic assay, fluorescence activated cell sorting (FACS), differential display, representational difference analysis, microarray, Western blotting, immunohistochemical staining, immunocytochemical staining, dot blots, or surface plasmon resonance detection. In another aspect, the liquid biological sample is selected from a saliva, a blood, a plasma, a serum, or a tear sample. In another aspect, the method further comprises the step of treating the patient negative for anti-Ro autoantibodies with a therapy that treats or reduces the symptoms of Sjögren's syndrome/Sjögren's disease.

As embodied and broadly described herein, an aspect of the present disclosure relates to an assay for detecting autoantibodies in anti-Ro antibody negative Sjögren's syndrome/Sjögren's disease comprising: obtaining a biological sample from a patient suspected of having an anti-Ro antibody negative Sjögren's disease; and detecting if the biological sample has autoantibodies to 1, 2, 3, 4, or 5, proteins selected from: GMNN, KLHDC8A, MAPRE1, NUP50, or SKIL, without performing a lip biopsy. In one aspect, the assay further comprises the steps of detecting if the biological sample has autoantibodies to at least one of SSB or SKIL, and determining that the patient has SS without performing a lip biopsy. In another aspect, the assay further comprises the step of detecting if the biological sample has autoantibodies to at least one additional biomarkers selected from ADP ribosylation factor GTPase activating protein 1 (ARFGAP1), Chromosome 9 Open Reading Frame 78 (C9orf78), Chromobox 3 (CBX3), Damage Specific DNA Binding Protein 1 (DDB1), GRB2 Associated Binding Protein 1 (GAB1), Heterogeneous Nuclear Ribonucleoprotein A/B (HNRNPAB), ISG15 Ubiquitin Like Modifier (ISG15), Multiple Coagulation Factor Deficiency 2 (MCFD2), NFU1 iron-sulfur cluster scaffold (NFU1), Pleckstrin Homology Domain Containing A4 (PLEKHA4), PML Nuclear Body Scaffold (PML), RNA Polymerase III Subunit H (POLR3H), POU Class 6 Homeobox 1 (POU6F1), RCAN Family Member 3 (RCAN3), SEC23 Interacting Protein (SEC23IP), SRY-box Transcription Factor 5 (SOX5), Small RNA Binding Exonuclease Protection Factor La (SSB), T-complex 10-like 3, pseudogene (TCP10L3), TPD52 Like 1 (TPD52L1), Triosephosphatase 1 (TPI1), Tripartite Motif Containing 21 (TRIM21), Ro60, Y RNA Binding Protein (TROVE2), Zinc Finger And BTB Domain Containing 46 (ZBTB46), and Zinc Finger Protein 655 (ZNF655). In another aspect, the autoantibodies are detected using an assay selected from at least one of: multiplex bead-based assay, capillary Western Blot, ELISA, flow cytometry, fluorimetry, microscopy, immunofluorescence, radioimmunoassay, immunoenzymatic assay, fluorescence activated cell sorting (FACS), differential display, representational difference analysis, microarray, Western blotting, immunohistochemical staining, immunocytochemical staining, dot blots, or surface plasmon resonance detection. In another aspect, the liquid biological sample selected from a saliva, a blood, a plasma, a serum, or a tear sample. In another aspect, the assay further comprises the step of treating the patient negative for Ro autoantibodies with a therapy that treats or reduces the symptoms of SS.

As embodied and broadly described herein, an aspect of the present disclosure relates to a kit comprising a synthetic or recombinant polypeptide covalently attached to a solid support, wherein the synthetic or recombinant polypeptide selected from: GMNN, KLHDC8A, MAPRE1, NUP50, or SKIL. In one aspect, the kit further comprises instructions for contacting the solid support with a biological sample from a patient suspected of having Sjögren's syndrome. In another aspect, the solid support is selected from the group consisting of a multiwell plate, an enzyme-linked immunosorbent assay (ELISA) plate, a microarray, a bead, a porous strip, and a nitrocellulose filter. In another aspect, the kit is an assay selected from the group consisting of a multiplex bead-based assay, capillary Western Blot, ELISA, flow cytometry, fluorimetry, microscopy, immunofluorescence, radioimmunoassay, immunoenzymatic assay, fluorescence activated cell sorting (FACS), differential display, representational difference analysis, microarray, Western blotting, immunohistochemical staining, immunocytochemical staining, dot blots, or surface plasmon resonance detection. In another aspect, the kit further comprises a secondary antibody labeled directly or indirectly with a detectable moiety. In another aspect, the kit further comprises the steps of detecting if the biological sample has autoantibodies to at least one of SSB or SKIL, and determining that the patient has SS without performing a lip biopsy. In another aspect, the kit further comprises at least one additional biomarkers selected from ADP ribosylation factor GTPase activating protein 1 (ARFGAP1), Chromosome 9 Open Reading Frame 78 (C9orf78), Chromobox 3 (CBX3), Damage Specific DNA Binding Protein 1 (DDB1), GRB2 Associated Binding Protein 1 (GAB1), Heterogeneous Nuclear Ribonucleoprotein A/B (HNRNPAB), ISG15 Ubiquitin Like Modifier (ISG15), Multiple Coagulation Factor Deficiency 2 (MCFD2), NFU1 iron-sulfur cluster scaffold (NFU1), Pleckstrin Homology Domain Containing A4 (PLEKHA4), PML Nuclear Body Scaffold (PML), RNA Polymerase III Subunit H (POLR3H), POU Class 6 Homeobox 1 (POU6F1), RCAN Family Member 3 (RCAN3), SEC23 Interacting Protein (SEC23IP), SRY-box Transcription Factor 5 (SOX5), Small RNA Binding Exonuclease Protection Factor La (SSB), T-complex 10-like 3, pseudogene (TCP10L3), TPD52 Like 1 (TPD52L1), Triosephosphatase 1 (TPI1), Tripartite Motif Containing 21 (TRIM21), Ro60, Y RNA Binding Protein (TROVE2), Zinc Finger And BTB Domain Containing 46 (ZBTB46), Zinc Finger Protein 655 (ZNF655).

As embodied and broadly described herein, an aspect of the present disclosure relates to a method of determining that a patient negative for Ro autoantibodies has Sjögren's syndrome (SS) without performing a lip biopsy comprising: obtaining a liquid biological sample from the patient suspected of having SS; determining, by a computer device, that the patient is negative for Ro autoantibodies; and detecting, by a computer device, if the biological sample has autoantibodies to 1, 2, 3, 4, or 5, proteins selected from: GMNN, KLHDC8A, MAPRE1, NUP50, or SKIL. In one aspect, the method further comprises the steps of detecting if the biological sample has autoantibodies to at least one of SSB or SKIL, and determining that the patient has SS without performing a lip biopsy. In another aspect, the method further comprises the step of detecting if the biological sample has autoantibodies to at least one additional biomarkers selected from ADP ribosylation factor GTPase activating protein 1 (ARFGAP1), Chromosome 9 Open Reading Frame 78 (C9orf78), Chromobox 3 (CBX3), Damage Specific DNA Binding Protein 1 (DDB1), GRB2 Associated Binding Protein 1 (GAB1), Heterogeneous Nuclear Ribonucleoprotein A/B (HNRNPAB), ISG15 Ubiquitin Like Modifier (ISG15), Multiple Coagulation Factor Deficiency 2 (MCFD2), NFU1 iron-sulfur cluster scaffold (NFU1), Pleckstrin Homology Domain Containing A4 (PLEKHA4), PML Nuclear Body Scaffold (PML), RNA Polymerase III Subunit H (POLR3H), POU Class 6 Homeobox 1 (POU6F1), RCAN Family Member 3 (RCAN3), SEC23 Interacting Protein (SEC23IP), SRY-box Transcription Factor 5 (SOX5), Small RNA Binding Exonuclease Protection Factor La (SSB), T-complex 10-like 3, pseudogene (TCP10L3), TPD52 Like 1 (TPD52L1), Triosephosphatase 1 (TPI1), Tripartite Motif Containing 21 (TRIM21), Ro60, Y RNA Binding Protein (TROVE2), Zinc Finger And BTB Domain Containing 46 (ZBTB46), Zinc Finger Protein 655 (ZNF655). In another aspect, the autoantibodies are detected using an assay selected from at least one of: multiplex bead-based assay, capillary Western Blot, ELISA, flow cytometry, fluorimetry, microscopy, immunofluorescence, radioimmunoassay, immunoenzymatic assay, fluorescence activated cell sorting (FACS), differential display, representational difference analysis, microarray, Western blotting, immunohistochemical staining, immunocytochemical staining, dot blots, or surface plasmon resonance detection. In another aspect, the liquid biological sample is selected from a saliva, a blood, a plasma, a serum, or a tear sample. In another aspect, the method further comprises the step of treating the patient negative for Ro autoantibodies with a therapy that treats or reduces the symptoms of SS.

### BRIEF DESCRIPTION OF THE DRAWINGS

For a more complete understanding of the features and advantages of the present invention, reference is now made to the detailed description of the invention along with the accompanying figures and in which:

FIG. 1 is a Venn diagram that shows the Canonical and novel antigens bound in plasma of Ro positive, Ro negative, and/or Other Disease groups at 3 SD above the mean of the healthy control group and Fisher's Exact Test p<0.05 compared to the healthy control group.

FIG. 2 is a Venn diagram that shows the Canonical and novel antigens bound in plasma of Ro positive, Ro negative, and/or Other Disease groups at 3 SD above the mean of the healthy control group and Fisher's Exact Test p<0.1 compared to the healthy control group.

FIGS. 3A and 3B shows the binding of novel and canonical antigens by plasma and saliva Ig of anti-Ro positive and anti-Ro negative cases of validation dataset. (A) Plasma, (B) Stimulated parotid saliva. White indicates specificities with normalized intensity values above the positive threshold (mean+3SD) of HC values. Gray indicates saliva samples not available or excluded due to high background. In each figure, upper panels indicate specificities significantly bound by SS cases, lower panels indicate specificities significantly bound by OD controls only (p<0.1, Fisher's exact test, gene symbols used to refer to protein products).

FIG. 4 shows capillary western blot of plasma IgG binding SOX5, FUT8, and GMNN. Binding of patient IgG antibodies to select proteins (yeast expressed, CDI). Commercial monoclonal (SOX5) or polyclonal (FUT8, GMNN) antibodies were used as positive controls (PC). Capillaries were loaded with 0.2 g/mL (SOX5) or 10 g/mL (FUT8, GMNN) protein solutions. Subject IDs and plasma dilutions listed at the top of each panel. HC=healthy control. Gene symbols used to refer to protein products.

FIGS. 5A and 5B show the top 30 antigens (including Ro60/TROVE2, Ro52/TRIM21 and La/SSB) useful for distinguishing between anti-Ro negative Sjögren's disease cases and healthy controls as determined by random forest machine learning analysis. Features (specificities) selected by random forest analyses for predictive model of SS using the validation dataset (FIG. 5A) including Ro and La proteome array binding data and (FIG. 5B) excluding Ro and La proteome array binding data. Presence of features in discovery and/or validation dataset(s) indicated with X.

FIG. 6A shows receiver operator characteristic (ROC) analysis employing the antigens/features presented in FIG. 5 that were derived from random forest machine learning using ⅔ of the custom proteome array validation dataset for training and ⅓ of the custom proteome array validation dataset for testing.

FIG. 6B shows receiver operator characteristic (ROC) analysis employing the antigens/features presented in FIG. 7 derived from random forest machine learning using ⅔ of the custom proteome array validation dataset for training and ⅓ of the custom proteome array validation dataset for testing.

FIG. 7 shows the binding of novel and canonical antigens by plasma Ig of anti-Ro positive and anti-Ro negative cases from an independent rheumatology practice cohort. Heatmap indicates specificities with normalized intensity values above the positive threshold (mean+3SD of HC values, green).

FIG. 8A to 8C show the clinical correlations with non-canonical antibody specificities. SS validation cohort patients with plasma Ig binding to at least 1 of 30 antigens identified by machine learning (FIGS. 6A and 6B) exhibit increased serum IgM levels which were found to be elevated in Ro− patients (FIG. 8A), but not in Ro+ patients (FIG. 8B). Ro+SS patients with Ig binding to MR5 exhibited more severe SS by multiple measures (FIG. 8C). (Mann-Whitney or Fisher's exact tests, p<0.05).

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity but include the general class of which a specific example may be used for illustration. The terminology herein is used to describe specific embodiments of the invention, but their usage does not limit the invention, except as outlined in the claims.

Abbreviations. ADP ribosylation factor GTPase activating protein 1 (ARFGAP1), Chromosome 9 Open Reading Frame 78 (C9orf78), Chromobox 3 (CBX3), CROCC Pseudogene 2 (CROCCP2), Damage Specific DNA Binding Protein 1 (DDB1), EGF like Fibronectin type III and Laminin G Domains (EGFLAM), Fucosyltransferase 8 (FUT8), GRB2 Associated Binding Protein 1 (GAB1), Geminin DNA Replication Inhibitor (GMNN), GRAM Domain Containing 1A (GRAMD1A), Heterogeneous Nuclear Ribonucleoprotein A/B (HNRNPAB), ISG15 Ubiquitin Like Modifier (ISG15), Kelch Domain Containing 8A (KLHDC8A), Microtubule Associated Protein RP/EB Family Member 1 (MAPRE1), Multiple Coagulation Factor Deficiency 2 (MCFD2), ER Cargo Receptor Complex Subunity (MCFD2), NFU1 iron-sulfur cluster scaffold (NFU1), Nucleoporin 50 (NUP50), 3-Phosphoinositide Dependent Protein Kinase 1 (PDPK1), Pleckstrin Homology Domain Containing A4 (PLEKHA4), PML Nuclear Body Scaffold (PML), RNA Polymerase III Subunit H (POLR3H), POU Class 6 Homeobox 1 (POU6F1), RCAN Family Member 3 (RCAN3), RNA Polymerase II Associated Protein 3 (RPAP3), SEC23 Interacting Protein (SEC23IP), SKI Like Proto-Oncogene (SKIL), SRY-box Transcription Factor 5 (SOX5), SRY-box Transcription Factor 13 (SOX13), T-complex 10-like 3, pseudogene (TCP10L3), TPD52 Like 1 (TPD52L1), Triosephosphatase 1 (TPI1), Tripartite Motif Containing 21 (TRIM21), Ro60, Y RNA Binding Protein (TROVE2), Zinc Finger And BTB Domain Containing 46 (ZBTB46), Zinc Finger Protein 655 (ZNF655).

Sjögren's syndrome, also known as Sjögren's disease (SjD), is a rheumatic autoimmune disease selectively targeting salivary and lacrimal glands, leading to painful dry mouth and eyes, oral infections, severe dental caries/tooth loss, fatigue, arthritis, nervous system involvement, and malignant B cell lymphoma. Current internationally accepted disease classification criteria rely on either the presence of anti-Ro antibodies (these may target either the Ro60 antigen, Ro52 antigen, or both) or the presence of focal lymphocytic infiltrates in a salivary gland lip biopsy for diagnosis. Either one of these features, in combination with one or more objective dryness measures, is necessary for the fulfillment of classification criteria for SS.

As used herein, the term “biomarker” or “biomarkers” refer to one or more characteristics that are objectively measured and evaluated as indicators of a normal or abnormal biological process, pathogenic (disease) processes, or pharmacologic responses to therapeutic interventions. As used in the context of Sjögren's syndrome patients, the biomarkers are auto-antibodies that target certain proteins as shown herein.

As used herein, the terms “detectable”, “detectable biomarkers”, or “detectable labels” are used interchangeably to refer to directly or indirectly detecting a compound or composition that is conjugated directly or indirectly to the composition to be detected, e.g., a protein, element, or other molecule, such as an antibody or enzyme to generate a “labeled” composition. Detectable compounds and/or elements can be detected due to their specific functional properties and/or chemical characteristics, the use of which allows the agent to which they are attached or attachable to be detected, and/or further quantified if desired, such as, e.g., an enzyme, radioisotope, electron dense particles, magnetic particles or chromophore. There are many types of detectable labels, including fluorescent labels, which are easily handled, inexpensive and nontoxic. The detectable portion can be attached to, e.g., an antibody that is specific for human antibodies, such that it forms a sandwich with the antigens, e.g., a sandwich ELISA or other secondary binding of agents to one or more detectable labels.

As used herein, the term “treating” refers to curing as well as ameliorating at least one symptom of Sjögren's syndrome.

As used herein, the term “effective amount” refers to the amount of a compound or agent administered or delivered to the patient which is most likely to result in the desired treatment outcome. The amount is empirically determined by the patient's clinical parameters including, but not limited to the stage of disease, age, gender, histology, and likelihood for recurrence.

The present inventors have discovered and validated panels of proteins useful for detecting autoantibodies in Sjögren's syndrome patients who lack anti-Ro/SS-A autoantibodies. Up to 40% of Sjögren's syndrome patients meeting classification criteria for this disorder lack antibodies to Ro/SS-A and must have minor salivary gland lip biopsy to confirm diagnosis. The novel panel of autoantigens can be used to detect anti-Ro negative Sjögren's disease without a lip biopsy.

The inventors constructed custom proteome arrays containing 150 antigens based on initial screenings using full proteome arrays (containing 15,500-19,500 human proteins). Samples from much larger numbers of Sjögren's disease cases, healthy controls, and other disease controls, were screened. Most (about 85%) antigens bound by Ro antibody negative Sjögren's patients, but not by healthy controls in the follow-up Validation Dataset, were also identified in the initial Discovery Dataset and are thus independently validated. Additional validation experiments using 10 of the antigens were conducted using the independent method of capillary Western blot. To date, validated reactivity to SOX5 and FUT8 was confirmed by, e.g., capillary Western blot.

The inventors conducted an unbiased screen of intact proteins covering a very large portion of the human proteome to look for previously undiscovered autoantibodies in Sjögren's syndrome/disease, with a primary focus on anti-Ro antibody negative Sjögren's.

Reactivity to at least one antigen in our panel identifies Ro antibody negative Sjögren's cases with 100% specificity and approximately 50% sensitivity. Therefore, the present invention enables diagnosis of about half of Ro antibody negative Sjögren's cases without a minor salivary gland lip biopsy. Minor salivary gland lip biopsy is not readily available in most clinical settings. The novel autoantibody panel can be included along with other blood work (such as ANA, Ro/SS-A, La-SSB) and clinical tests to enable diagnosis of Ro antibody negative Sjögren's without a lip biopsy. Reactivity to at least one antigen in the panel identifies Ro antibody negative Sjögren's cases with 100% specificity and approximately 50% sensitivity.

Identification of candidate novel autoantigens by screening human proteome arrays. Proteins from >15,500-19,500 human genes (nearly 90% are full length), which is 75% coverage of human proteome, that are yeast-expressed and purified with N-terminal GST and 6×His tags. The proteins are spotted in duplicate on ultrathin nitrocellulose-coated glass slides.

To evaluate binding to the >15,500-19,500 human genes for candidate autoantigen selection, plasma (n=21 Ro positive SjD cases, n=20 Ro negative SjD cases) and stimulated parotid saliva (n=5 Ro positive SjD cases, n=6 Ro negative SjD cases) were tested, as well as salivary gland plasmablast-derived mAbs (n=83, pooled 3-10 mAbs/array) from n=6 Ro positive SjD cases and n=3 Ro negative SjD cases. Healthy control samples tested included plasma from n=17 healthy control subjects, stimulated parotid saliva from n=1 healthy control subject, and 5 mAbs of irrelevant specificity from n=2 anthrax or rabies vaccine recipients. From this Discovery Dataset, 150 proteins were selected for printing custom arrays. Custom arrays were then used to test the following independent samples: plasma and stimulated parotid saliva from n=46 Ro positive SjD cases, n=50 Ro negative SjD cases, n=42 healthy controls, and n=54 Other Disease controls). These data collected from the custom arrays form the Validation Dataset.

Blood plasma of cases and controls were tested at 1:500 dilution (all age, race, and sex-matched). Stimulated parotid saliva of cases and controls were tested at 1:20 dilution. Data normalization was as follows: (1) Bioconductor Package in R, (2) Quantile Normalization, (3) all arrays normalized to 3 duplicate pairs of control IgG proteins on each array, and (4) correction for uneven printing effects.

Data analysis. A threshold of mean+3 SD of HC value for each protein was used, followed by Fisher's Exact Test to distinguish the evaluated patient groups from the Healthy Control group at p<0.1 or p<0.05.

Demographics. All groups were age, race, and sex-matched (not HLA). All participants were from the Oklahoma Sjögren's Research Clinic (except for n=9 individuals with multiple sclerosis that were part of the Other Disease group). All SjD cases met the 2002 American-European Consensus Criteria (AECG) classification criteria for Sjögren's syndrome, and a subset of cases also met the 2016 American College of Rhematology/European League Against Rheumatism (ACR/EULAR) classification criteria for Sjögren's syndrome. All other disease subjects met currently accepted disease classification criteria.

TABLE 1
Demographics of subjects from the Validation Dataset.

| | HC | Ro Pos | Ro Neg | Other Disease |
|---|---|---|---|---|
| | (n = 42) | (n = 46) | (n = 50) | (n = 54) |
| Age (Mean(SD)) | 50(14) | 49(14) | 50(14) | 49(14) |
| Race | | | | |
| White | 64% | 67% | 72% | 59% |
| NatAm/>one | 26% | 30% | 28% | 30% |
| Black/Asian | 2% | 2% | 0% | 6% |
| Sex | | | | |
| Female | 93% | 94% | 94% | 91% |
| Male | 7% | 6% | 6% | 9% |

White, self-reported European American descent
NatAm/>one, Native American and more than one race
Race data unavailable for 3 HC and 3 Other Disease

TABLE 2
Distribution of number of specificities per subject - Plasma.

| | HC | Ro Pos | Ro Neg | Other Disease |
|---|---|---|---|---|
| Plasma | (n = 42) | (n = 46) | (n = 50) | (n = 54) |
| 0-5 | 39 (93%) | 30 (65%) | 40 (80%) | 45 (83%) |
| 6-10 | 1 (2%) | 11 (24%) | 4 (8%) | 5 (9%) |
| >10 | 2 (5%) | 5 (11%) | 6 (12%) | 4 (7%) |

TABLE 3
Distribution of number of specificities per subject - Saliva.

| | HC | Ro Pos | Ro Neg | Other Disease |
|---|---|---|---|---|
| Saliva | (n = 42) | (n = 42) | (n = 47) | (n = 42) |
| 0-5 | 41 (98%) | 36 (86%) | 42 (89%) | 38 (90%) |
| 6-10 | 0 | 4 (10%) | 1 (2%) | 1 (2%) |
| >10 | 1 (2%) | 2 (5%) | 4 (9%) | 3 (7%) |

TABLE 4
Canonical and novel antigens were significantly bound in plasma.

| | HC | Ro Pos | Ro Neg | Other Disease |
|---|---|---|---|---|
| Plasma | (n = 42) | (n = 46) | (n = 50) | (n = 54) |
| TROVE2/Ro60 | 0% | 74% | 10% | 11% |
| TRIM21/Ro52 | 0% | 22% | 0% | 0% |
| SSB/La | 2% | 48% | 22% | 7% |
| CHRM5 | 2% | 52% | 6% | 2% |
| TCP10 | 0% | 13% | 4% | 7% |
| ZBTB46 | 0% | 13% | 10% | 9% |
| CROCCP2 | 0% | 11% | 4% | 9% |
| SKIL | 0% | 11% | 16% | 7% |
| NUP50 | 0% | 2% | 16% | 2% |
| GMNN | 0% | 7% | 14% | 9% |
| KLHDC8A | 0% | 2% | 14% | 6% |
| MAPRE1 | 0% | 4% | 14% | 6% |
| RCAN3 | 0% | 2% | 12% | 11% |
| GRAMD1A | 0% | 2% | 10% | 0% |
| ISG15 | 0% | 2% | 10% | 2% |
| POLR3H | 0% | 7% | 10% | 6% |
| RPAP3 | 0% | 2% | 10% | 2% |

p < 0.05,
0.05 < p < 0.1

TABLE 5
Canonical and novel antigens were significantly bound in saliva.

| | HC | Ro Pos | Ro Neg | Other Disease |
|---|---|---|---|---|
| Saliva | (n = 42) | (n = 42) | (n = 47) | (n = 42) |
| TROVE2/Ro60 | 2% | 52% | 9% | 10% |
| TRIM21/Ro52 | 0% | 33% | 0% | 5% |
| SSB/La | 2% | 29% | 6% | 10% |
| CHRM5 | 0% | 33% | 6% | 2% |

p < 0.05;
results were identical at p < 0.1

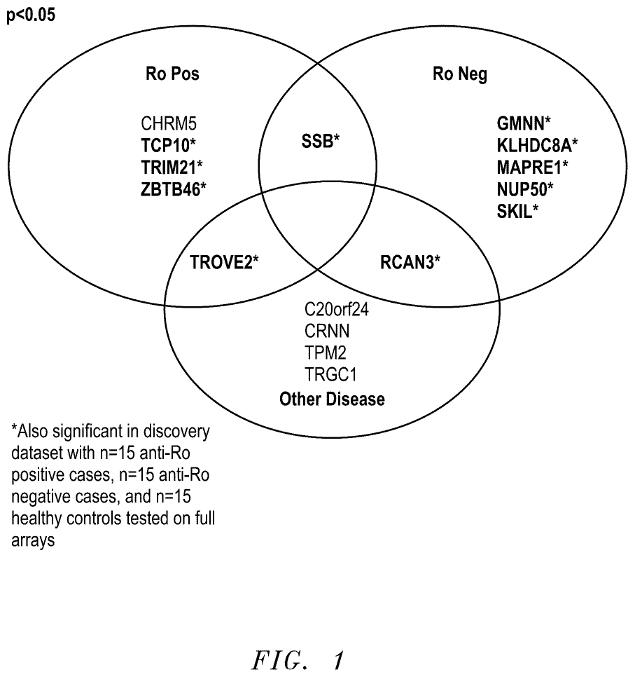

FIG. 1 is a Venn diagram indicating antigens bound by plasma Ig greater than the mean+3SD of the healthy control group and differing from the healthy control group by p<0.05 as assessed by Fisher's Exact Test. Results are from the validation dataset. Antigens with “*” were also independently identified as novel antigens using the same criteria in the discovery dataset.

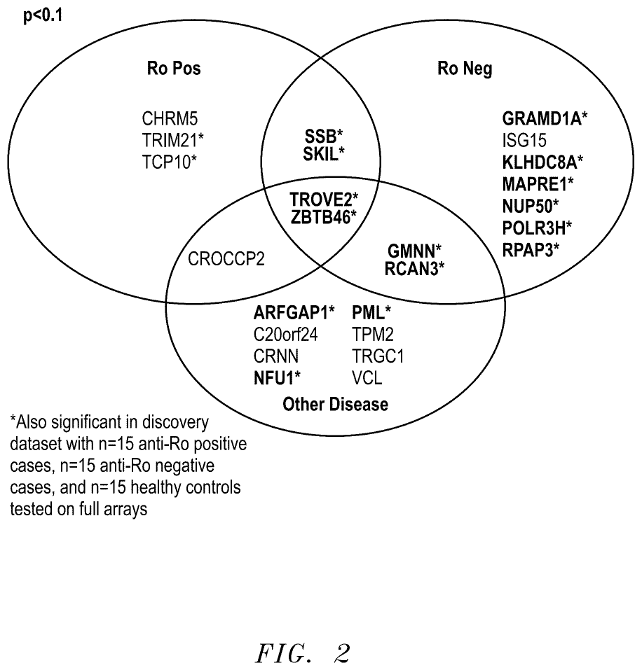

FIG. 2 is a Venn diagram indicating antigens bound by plasma Ig greater than the mean+3 SD of the healthy control group and differing from the healthy control group by p<0.1 as assessed by Fisher's Exact Test. Results are from the validation dataset. Antigens with “*” were also independently identified as novel antigens using the same criteria in the discovery dataset.

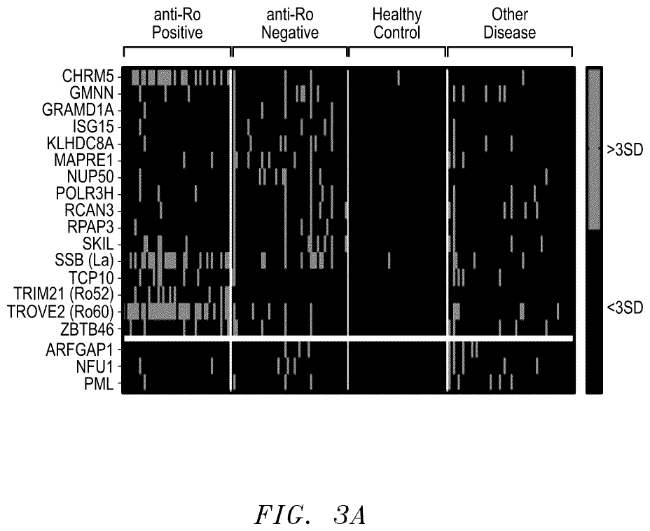

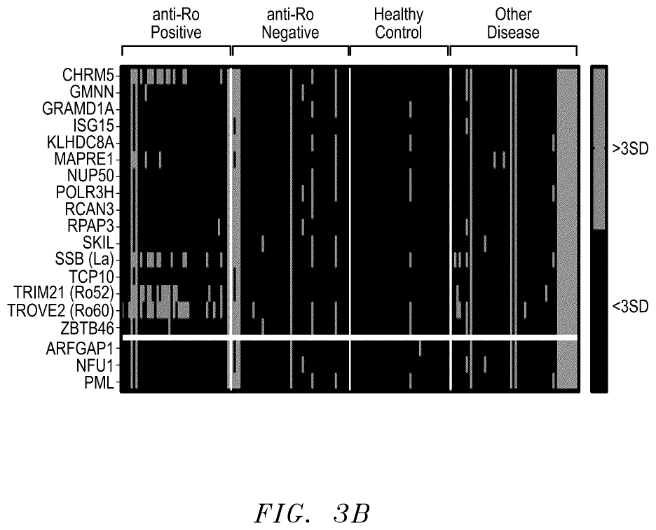

The majority of cases bound ≤10 specificities in plasma and saliva, with a mean of 3 and 4 in Ro+ and Ro− plasma, respectively, and a mean of 2 and 3 in Ro+ and Ro-saliva, respectively. Individual-level plasma antibody results, as well as clinical serologic test results for antibodies to the canonical SS antigens, are summarized in SI Table 5. Plasma antibodies of a few individuals bound to 10 or more antigens, which could reflect polyreactivity. In plasma, we identified 16 specificities excluding the Ro and La antigens, three of which were commonly bound by plasma Ig in the OD group only (ARFGAP1, NFU1, and PML) (FIG. 3A). Of the remaining 13 specificities, 11 (GMNN, GRAMD1A, KLHDC8A, MAPRE1, NUP50, POLR3H, RCAN3, RPAP3, SKIL, TCP10, and ZBTB46) were commonly bound by antibodies in ≥4 cases in the discovery dataset, while ISG15 was recognized by antibodies from 3 cases and enriched in the salivary gland, thus confirming these antibodies in larger groups of subjects. Except for 9 proteins, all antigens identified in the discovery dataset were bound by plasma Ig from at least one SS case, with more than 20 proteins bound by at least 5 SS cases. Surprisingly, five Ro− cases bound TROVE2 (Ro60) on the array, which may be due to differences in assay sensitivity or source species of proteins. Only the Ro and La antigens and MR5 were commonly bound by salivary Ig in SS cases (FIG. 3B).

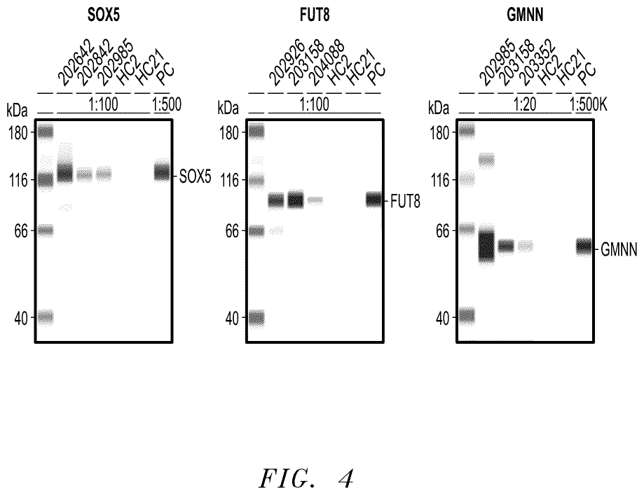

The inventors used capillary western blot to confirm the binding of plasma antibodies to select proteins, including 8 proteins commonly bound by the SS group in the validation dataset and/or identified by random forest analyses: CBX3, FUT8, GMNN, KLHDC8A, MAPRE1, NUP50, SKIL, and ZBTB46. Two other proteins (RPS29 and SOX5) selected from the discovery dataset did not replicate in the validation dataset when testing nine samples in parallel on both sets of arrays (data not shown), suggesting array-dependent differences in protein preparations. Each protein was tested with plasma from three subjects that bound the selected protein on the arrays and two HC that did not react to any of the selected proteins. With this assay, it was confirmed that plasma IgG antibodies to SOX5, FUT8, and GMNN, were found exclusively in the SS cases. (FIG. 4). HC=healthy control. Positive control (PC) antibodies were commercially sourced monoclonal or polyclonal antibodies (FUT8 is polyclonal mouse anti-human IgG, Novus Biologicals, catalog number H00002530-B01P; SOX5 is monoclonal mouse anti-human IgG, Novus Biologicals, catalog number NBP2-03766).

TABLE 6
Diagnostic potential of the panel of canonical (Ro60/TROVE2,
Ro52/TRIM21) and novel antigens (Fisher's Exact Test p <
0.1) bound by plasma IgG of SS/SjD cases compared with healthy
controls. Evaluation includes all SS/SjD cases meeting the 2002
revised American European Consensus Criteria for primary SS.

| | | | | | Other |
|---|---|---|---|---|---|
| | HC | Ro Pos | Ro Neg | Ro Pos & Ro | Disease |
| Plasma | (n = 42) | (n = 46) | (n = 50) | Neg (n = 96) | (n = 54) |
| Binds ≥1 Novel | 0% | 83% | 56% | 69% | 37% |
| Shared Ag | | | | | |
| Avg* | N/A | 2.1 | 2.8 | 2.4 | 2.4 |
| PPV | | | 100% | 100% | |
| NPV | | | 66% | 58% | |
| Sensitivity | | | 56% | 69% | |
| Specificity | | | 100% | 100% | |
| Accuracy | | | 76% | 78% | |

*average number of antigens bound per subject
PPV—probability of having disease w/pos result
NPV—probability of not having disease w/neg result
Sensitivity—probability of pos result in pt with disease
Specificity—probability of neg result in pt without disease
Accuracy—proportion of “true positives” and “true negatives” among all subjects

| | |
|---|---|
| BioPlex Comparison | Binds ≥1 Ro52/La/Ro60 |

| | | |
|---|---|---|
| Anti-Ro Positive (n = 46) | 46/46 | 100% |
| Anti-Ro Negative (n = 50) | 16/50 | 32% |
| Combined (n = 96) | 62/96 | 65% |

TABLE 7
Panel of novel antigens (Fisher's Exact Test p < 0.1)
bound by plasma IgG of SjD cases compared with controls. Canonical
antigens (Ro60/TROVE2, Ro52/TRIM21, La/SSB) were excluded.
Evaluation includes all SS/SjD cases meeting the 2002 revised
American European Consensus Criteria for primary SS.

| | | | | | Other |
|---|---|---|---|---|---|
| | HC | Ro Pos | Ro Neg | Ro Pos & Ro | Disease |
| Plasma | (n = 42) | (n = 46) | (n = 50) | Neg (n = 96) | (n = 54) |
| Binds ≥1 | 0% | 41% | 54% | 48% | 30% |
| Novel | | | | | |
| Shared Ag | | | | | |
| Avg* | N/A | 1.9 | 2.7 | 2.4 | 2.6 |
| PPV | | | 100% | 100% | |
| NPV | | | 65% | 46% | |
| Sensitivity | | | 54% | 48% | |
| Specificity | | | 100% | 100% | |
| Accuracy | | | 75% | 64% | |

*average number of antigens bound per subject
PPV—probability of having disease w/pos result
NPV—probability of not having disease w/neg result
Sensitivity—probability of pos result in pt with disease
Specificity—probability of neg result in pt without disease
Accuracy—proportion of “true positives” and “true negatives” among all subjects

| | |
|---|---|
| BioPlex Comparison | Binds ≥1 Ro52/La/Ro60 |

| | | |
|---|---|---|
| Anti-Ro Positive (n = 46) | 46/46 | 100% |
| Anti-Ro Negative (n = 50) | 16/50 | 32% |
| Combined (n = 96) | 62/96 | 65% |

TABLE 8
Diagnostic potential of the panel of canonical (Ro60/TROVE2,
Ro52/TRIM21) and novel antigens (Fisher's Exact Test p <
0.1) bound by plasma IgG of SS/SjD cases compared with healthy
controls. Evaluation includes subset of SS/SjD cases meeting
the 2016 ACR/EULAR classification criteria for SS/SjD.

| | | | | Ro Pos & | Other |
|---|---|---|---|---|---|
| | HC | Ro Pos | Ro Neg | Ro Neg | Disease |
| Plasma | (n = 42) | (n = 46) | (n = 35) | (n = 81) | (n = 54) |
| Binds ≥1 Novel | 0% | 83% | 49% | 68% | 37% |
| Shared Ag | | | | | |
| Avg* | N/A | 2.1 | 2.5 | 2.2 | 2.4 |
| PPV | | | 100% | 100% | |
| NPV | | | 70% | 62% | |
| Sensitivity | | | 49% | 68% | |
| Specificity | | | 100% | 100% | |
| Accuracy | | | 77% | 79% | |

*average number of antigens bound per subject
PPV—probability of having disease w/pos result
NPV—probability of not having disease w/neg result
Sensitivity—probability of pos result in pt with disease
Specificity—probability of neg result in pt without disease

| | |
|---|---|
| BioPlex Comparison | Binds ≥1 Ro52/La/Ro60 |

| | | |
|---|---|---|
| Anti-Ro Positive (n = 46) | 46/46 | 100% |
| Anti-Ro Negative (n = 35) | 3/35 | 9% |
| Combined (n = 81) | 49/81 | 60% |

TABLE 9
Panel of novel antigens (Fisher's Exact Test p < 0.1)
bound by plasma IgG of SjD cases compared with controls.
Canonical antigens (Ro60/TROVE2, Ro52/TRIM21, La/SSB) were
excluded. Evaluation includes subset of SjD cases meeting
the 2016 ACR/EULAR classification critera for Sjogren's syndrome.

| | | | | Ro Pos & | Other |
|---|---|---|---|---|---|
| | HC | Ro Pos | Ro Neg | Ro Neg | Disease |
| Plasma | (n = 42) | (n = 46) | (n = 35) | (n = 81) | (n = 54) |
| Binds ≥1 Novel | 0% | 41% | 46% | 43% | 30% |
| Shared Ag | | | | | |
| Avg* | N/A | 1.9 | 2.6 | 2.2 | 2.6 |
| PPV | | | 100% | 100% | |
| NPV | | | 69% | 48% | |
| Sensitivity | | | 46% | 43% | |
| Specificity | | | 100% | 100% | |
| Accuracy | | | 75% | 63% | |

*average number of antigens bound per subject
PPV—probability of having disease w/pos result
NPV—probability of not having disease w/neg result
Sensitivity—probability of pos result in pt with disease
Specificity—probability of neg result in pt without disease

| | |
|---|---|
| BioPlex Comparison | Binds ≥1 Ro52/La/Ro60 |

| | | |
|---|---|---|
| Anti-Ro Positive (n = 46) | 46/46 | 100% |
| Anti-Ro Negative (n = 35) | 3/35 | 9% |
| Combined (n = 81) | 49/81 | 60% |

TABLE 10
STRING analysis reveals pathways targeted by
autoimmune response in anti-Ro negative SjD.

| | | #observed | | |
|---|---|---|---|---|
| Pathway | #novel Ag | genes | Strength | FDR |
| Leukemia cell | 8 | 12 | 0.77 | 0.00097 |
| Ubiquitin mediated | 2 | 5 | 1.24 | 0.002 |
| proteolysis | | | | |
| Post-translational protein | 7 | 13 | 0.64 | 0.0095 |
| modification | | | | |
| ubiquitin ligase | 11 | 15 | 0.47 | 0.0256 |
| conjugation | | | | |
| Antiviral defense | 3 | 4 | 1.17 | 0.0394 |
| TGF-beta signaling pathway | 2 | 4 | 1.15 | 0.0451 |

Protein interactions and tissue expression of specificities determined with STRING and Protein Atlas. Protein interactions and expression of the antigens were explored using the STRING and Human Protein Atlas databases, respectively. Important specificities from the random forest analyses, as well as those commonly recognized by the SS, but not HC or OD groups (FIGS. 3A and 3B), were entered into the STRING database to uncover pathways enriched for the identified antigens. Analyses were performed with and without, the Ro and La autoantigens. Table 11 shows that leukemia cell, ubiquitin conjugation, and antiviral defense pathways were among the most significant results in both analyses. A query of the Human Protein Atlas revealed biased expression of certain antigens in particular tissues or cells, with EGFLAM, ISG15, TPD52L1, and TPI1 being enriched in salivary gland or tongue and RCAN3 involved in oral antimicrobial defense. Other proteins showed expression in particular cell types of the immune system, brain, testis, prostate, ovary, or pancreas.

TABLE 11
STRING Interactome Analysis.

| Category | Term description | FDR | Antigens |
|---|---|---|---|
| TISSUES | Leukemia cell | [symbol] | [symbol] |
| KEGG | Ubiquitin mediated proteolysis | 0.002 | [symbol] |
| Reactome | Post-translational | [symbol] | [symbol] |
| | protein modification | | |
| UniProt | Ubiquitin conjugation | [symbol] | [symbol] |
| Keywords | | | |
| UniProt | Antiviral defense | [symbol] | [symbol] |
| Keywords | | | |
| WikiPathways | TGF-beta signaling | 0.0451 | [symbol] |
| | pathway | | |

Novel specificities are indicated in bold italics. Only pathways including at least two novel specificities are shown. [symbol]
indicates data missing or illegible when filed

The present inventors set out to identify serum/plasma autoantibodies in patients without anti-Ro antibodies. The present inventors identified novel specificities in anti-Ro antibody positive and anti-Ro antibody negative SjD cases, including antigens shared between the two groups.

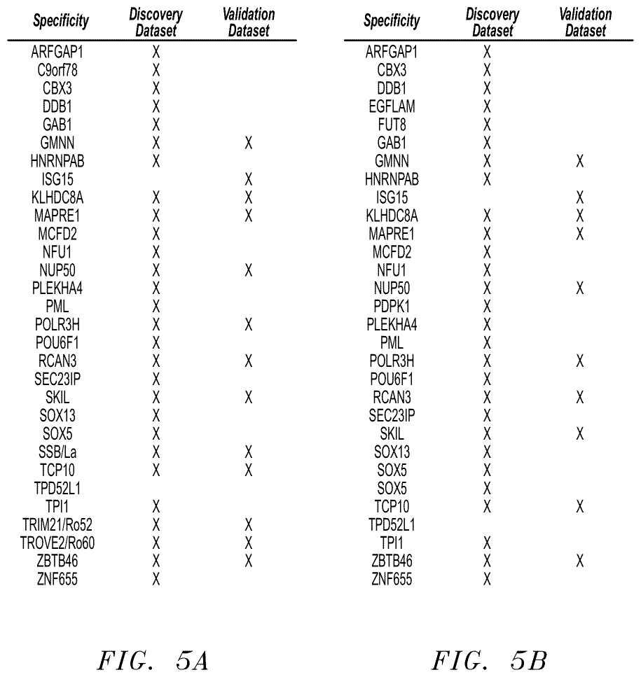

FIGS. 5A and 5B show the top 30 antigens (including Ro60/TROVE2, Ro52/TRIM21 and La/SSB) useful for distinguishing between anti-Ro negative Sjögren's disease cases and healthy controls as determined by random forest machine learning analysis. Features (specificities) selected by random forest analyses for predictive model of SS using the validation dataset (FIG. 5A) including Ro and La proteome array binding data and (FIG. 5B) excluding Ro and La proteome array binding data. Presence of features in discovery and/or validation dataset(s) indicated with X.

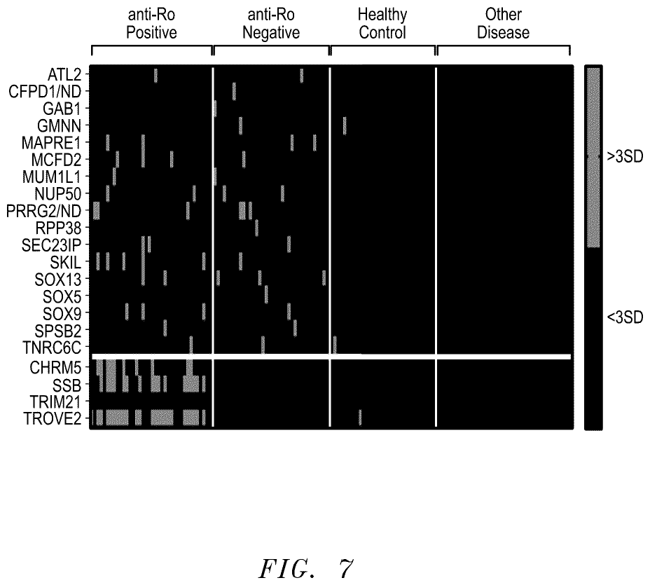

In both analyses, thirty features were identified that could predict SS (FIG. 4 and FIGS. 7A and 7B), several of which were also commonly recognized in the discovery dataset. Ro− SS could be distinguished from HC and OD controls with an area under the curve (AUC) of 0.88 (95% CI 0.78-0.96) with Ro and La array binding data and AUC of 0.79 (95% CI 0.64-0.93) without the canonical antigens. Unsurprisingly, the heterogeneous OD group could not be predicted as an entity.

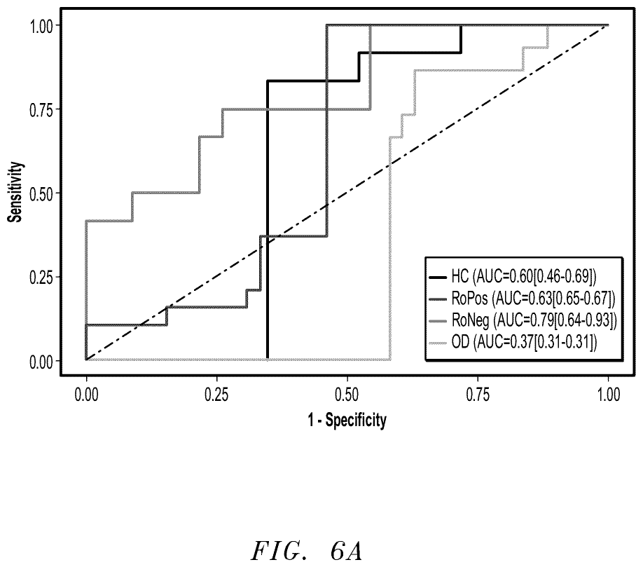

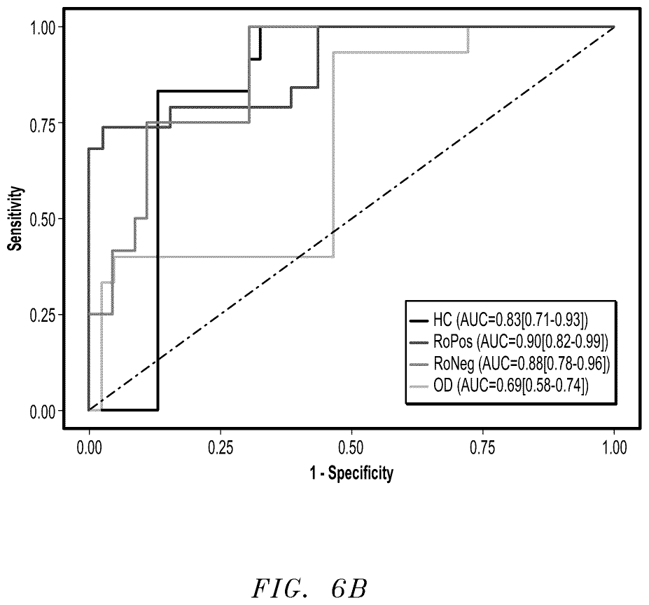

Assessment of an independent rheumatology practice cohort. Next, the inventors assessed applicability of the discovered non-canonical specificities to previously described rheumatology practice cohort (Table 2) by screening serum antibodies with the custom arrays. Individual-level results, as well as clinical serologic test results for antibodies to the canonical SS antigens. The independent JHU Ro− case group (n=36) had serum antibodies to 17 of the 70 non-canonical antigens, compared to binding to only 2 of the 70 antigens in 10 HC from the same site (χ²=13.7, p=0.0002; FIG. 5). Of the 17 non-canonical antigens bound, 9 were among those identified by random forest machine learning to predict the Ro− cases in the validation cohort shown in FIG. 6 and FIG. 7B. The independent Ro+ case group (n=38) had serum antibodies binding to 39 of the 70 non-canonical antigens compared to binding to 6 of the 70 antigens by 10 HC from the same site (×²=35.6, p<0.0001; FIG. 5). On an individual case basis, 50% (18 of 36) of the independent Ro− case group had serum antibodies binding to one or more of the 17 non-canonical antigens recognized by this group, similar to the frequency with which non-canonical antigens identify the Ro− validation cohort, while 2 of 10 independent HC bound at least one of the antigens (p=0.15, none of the antigens recognized by the JHU cases were more commonly bound compared to the JHU HC group, as defined by Fisher's exact p<0.1 Fisher's exact test). Of the n=10 JHU and n=42 OMRF HC cohorts combined, 12 had serum or plasma antibodies binding to at least one of the 17 antigens, distinguishing HC from the JHU Ro− cases (p=0.012). Comparison of the frequency with which the independent Ro+ cases recognized one or more of the 17 non-canonical antigens (39%) to the combined HC group did not distinguish the groups (p=0.108). However, the frequency with which the independent Ro+ cases recognized one or more of the 39 non-canonical antigens identified by that group (55%) did differ from that of the combined HC group (29%; p=0.016). When considered individually, none of the antigens recognized by the JHU cases were more commonly bound compared to the JHU HC group, as defined by Fisher's exact p<0.1.

Application of the random forest models described in FIG. 4B and FIG. 7B failed to predict the JHU Ro− case group (not shown), indicating differences in non-canonical antigen recognition between the cohorts. However, training of a new random forest machine learning model using ⅔ of the JHU SS cases and the combined JHU and OMRF HC, followed by testing on ⅓ of the independent cases and combined HC in the absence of Ro and La proteome array binding data resulted in a measurable and detectable predictive power for the JHU Ro− cases (ROC AUC 0.61 (95% CI 0.31-0.58)) that was further enhanced by the inclusion of the canonical Ro60, Ro52 and La binding data (ROC AUC 0.71 (95% CI 0.37-0.62)). Thus, while an independent SS cohort had serum antibodies to some of the newly discovered non-canonical antigens, they were not sufficient to predict SS status, and further discovery studies incorporating multiple SS cohorts are required.

FIG. 6A shows the Receiver Operating Characteristic (ROC) curve illustrating the capacity of 30 novel antigenic specificities identified by random forest machine learning in ⅔ of the validation dataset to distinguish the Ro negative group from the healthy control group in ⅓ of the validation dataset. Analysis used positive/negative binary values as determined from mean+3SD healthy control group thresholds.

FIG. 6B shows the Receiver Operating Characteristic (ROC) curve illustrating the capacity of 30 novel antigenic specificities identified by random forest machine learning in ⅔ of the validation dataset to distinguish Ro positive SjD, Ro negative SjD, Other Disease and healthy control groups from each other in ⅓ of the validation dataset. Canonical Sjögren's antigens were included in this analysis. Analysis used positive/negative binary values as determined from mean+3SD healthy control group thresholds.

FIG. 7 shows the binding of novel and canonical antigens by plasma Ig of anti-Ro positive and anti-Ro negative cases from an independent rheumatology practice cohort. Heatmap indicates specificities with normalized intensity values above the positive threshold (mean+3SD of HC values, green).

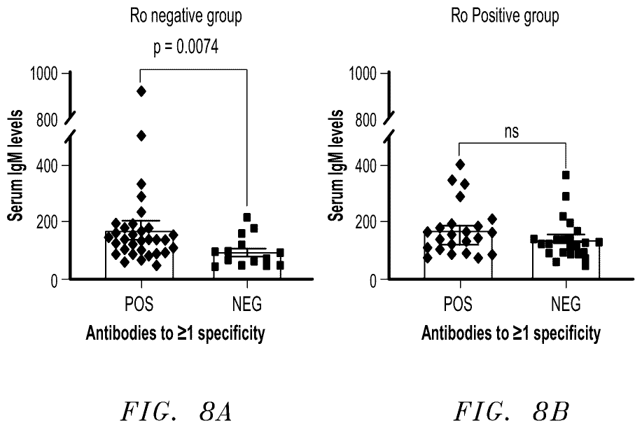

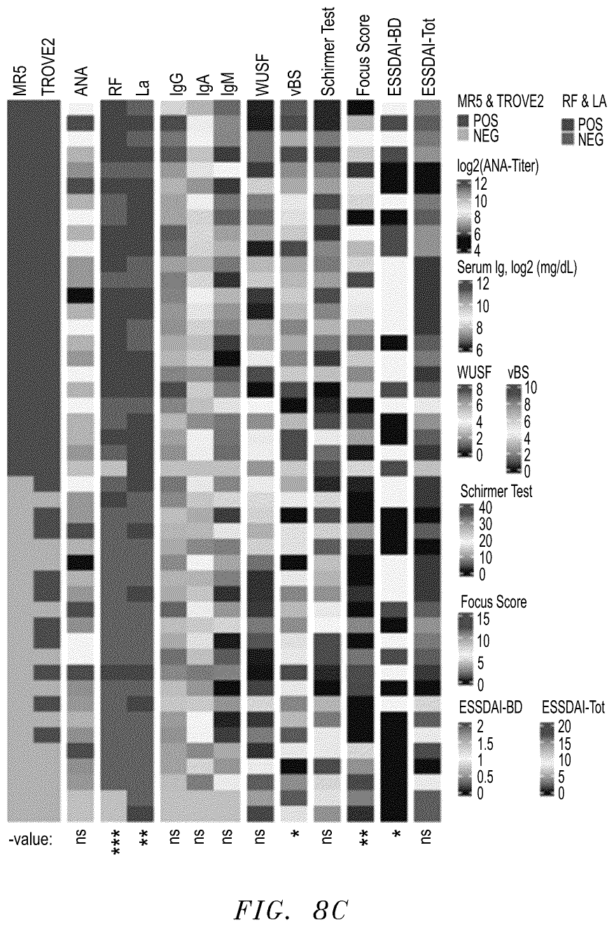

FIG. 8A to 8C show the clinical correlations with non-canonical antibody specificities. SS validation cohort patients with plasma Ig binding to at least 1 of 30 antigens identified by machine learning (FIGS. 6A and 6B) exhibit increased serum IgM levels which were found to be elevated in Ro− patients (FIG. 8A), but not in Ro+ patients (FIG. 8B). Ro+SS patients with Ig binding to MR5 exhibited more severe SS by multiple measures (FIG. 8C). (Mann-Whitney or Fisher's exact tests, p<0.05).

The present inventors set out to improve the detection of Sjögren's syndrome/Sjögren's disease in anti-Ro antibody negative cases. It was found that novel shared antigens may be new biomarkers to aid in diagnosis of SjD without a lip biopsy, especially for anti-Ro antibody negative cases.

A person of skill in the art would readily recognize that steps of various above-described methods can be performed by programmed computers. Herein, some embodiments are also intended to cover program storage devices, e.g., digital data storage media, which are machine or computer-readable and encode machine-executable or computer-executable programs of instructions, wherein said instructions perform some or all of the steps of said above-described methods. The program storage devices may be, e.g., digital memories, magnetic storage media such as a magnetic disk and/or magnetic tape, hard drives, or optically readable digital data storage media. The embodiments are also intended to cover computers programmed to perform said steps of the above-described methods.

The functions of the various elements shown in the figures, including any functional blocks labelled as “modules”, may be provided through the use of dedicated hardware as well as hardware capable of executing software in association with appropriate software. When provided by a processor, the functions may be provided by a single dedicated processor, by a single shared processor, or by a plurality of individual processors, some of which may be shared. Moreover, explicit use of the term “module” should not be construed to refer exclusively to hardware capable of executing software, and may implicitly include, without limitation, digital signal processor (DSP) hardware, network processor, application specific integrated circuit (ASIC), field programmable gate array (FPGA), read only memory (ROM) for storing software, random access memory (RAM), and non-volatile storage. Other hardware, conventional and/or custom, may also be included.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited features, elements, components, groups, integers, and/or steps, but do not exclude the presence of other unstated features, elements, components, groups, integers and/or steps. In embodiments of any of the compositions and methods provided herein, “comprising” may be replaced with “consisting essentially of” or “consisting of”. As used herein, the term “consisting” is used to indicate the presence of the recited integer (e.g., a feature, an element, a characteristic, a property, a method/process step or a limitation) or group of integers (e.g., feature(s), element(s), characteristic(s), property(ies), method/process steps or limitation(s)) only. As used herein, the phrase “consisting essentially of” requires the specified features, elements, components, groups, integers, and/or steps, but do not exclude the presence of other unstated features, elements, components, groups, integers and/or steps as well as those that do not materially affect the basic and novel characteristic(s) and/or function of the claimed invention.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least 1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

1: Danda D, Sharma R, Truong D, Koelsch K A, Kurien B T, Bagavant H, Deshmukh U, Kaufman C E, Lewis D M, Stone D U, Radfar L, Rasmussen A, Sivils K L, Scofield R H. Anti-La positive, anti-Ro negative subset of primary Sjögren's syndrome: anti-La is a reality but is the
2: Toker E, Yavuz S, Direskeneli H. Anti-Ro/SSA and anti-La/SSB autoantibodies in the tear fluid of patients with Sjögren's syndrome. Br J Ophthalmol. 2004 March; 88(3):384-7. doi: 10.1136/bjo.2003.028340. PMID: 14977774; PMCID: PMC1772044.
3: Retamozo S, Akasbi M, Brito-Zerón P, Bosch X, Bove A, Perez-de-Lis M, Jimenez I, Soto-Cardenas M J, Gandia M, Diaz-Lagares C, Viñas O, Siso A, Perez-Alvarez R, Yague J, Ramos-Casals M. Anti-Ro52 antibody testing influences the classification and clinical characterisation of primary Sjögren's syndrome. Clin Exp Rheumatol. 2012 September-October; 30(5):686-92. Epub 2012 Oct. 17. PMID: 22704838.
4: Hubschman S, Rojas M, Kalavar M, Kloosterboer A, Sabater A L, Galor A. Association Between Early Sjögren Markers and Symptoms and Signs of Dry Eye. Cornea. 2020 March; 39(3):311-315. doi: 10.1097/ICO.0000000000002171. PMID: 31634227; PMCID: PMC7007845.
5: Liu Y, Zhang B, Lei Y, Xia L, Lu J, Shen H. Serum levels of interleukin-34 and clinical correlation in patients with primary Sjögren's syndrome. Int J Rheum Dis. 2020 March; 23(3):374-380. doi: 10.1111/1756-185X.13773. Epub 2019 Dec. 10. PMID: 31823505.
6: Stone D U, Fife D, Brown M, Earley K E, Radfar L, Kaufman C E, Lewis D M, Rhodus N L, Segal B M, Wallace D J, Weisman M H, Venuturupalli S, Brennan M T, Lessard C J, Montgomery C G, Scofield R H, Sivils K L, Rasmussen A. Effect of Tobacco Smoking on The Clinical, Histopathological, and Serological Manifestations of Sjögren's Syndrome. PLoS One. 2017 Feb. 6; 12(2):e0170249. doi:10.1371/journal.pone.0170249. PMID: 28166540; PMCID: PMC5293551.
7: Menor Almagro R, Jurado Roger A, Rodriguez Gutierrez F J, Solis Diaz R, Cardiel M H, Salaberri Maestrojuan J J. Association of anti-Ro52, anti-Ro60 and anti-La antibodies with diagnostic, clinical and laboratory features in a referral hospital in Jerez, Spain. Reumatol Clin. 2016 September-October; 12(5):256-62. English, Spanish. doi: 10.1016/j.reuma.2015.10.010. Epub 2015 Dec. 25. PMID: 26725021.
8: Ter Borg E J, Kelder J C. Is extra-glandular organ damage in primary Sjögren's syndrome related to the presence of systemic auto-antibodies and/or hypergammaglobulinemia?A long-term cohort study with 110 patients from the Netherlands. Int J Rheum Dis. 2017 July; 20(7):875-881. doi:10.1111/1756-185X.13070. Epub 2017 Apr. 26. PMID: 28447402.
9: Brito-Zerón P, Acar-Denizli N, Ng W F, Zeher M, Rasmussen A, Mandl T, Seror R, Li X, Baldini C, Gottenberg J E, Danda D, Quartuccio L, Priori R, Hernandez-Molina G, Armagan B, Kruize A A, Kwok S K, Kvarnström M, Praprotnik S, Sène D, Bartoloni E, Solans R, Rischmueller M, Suzuki Y, Isenberg D A, Valim V, Wiland P, Nordmark G, Fraile G, Bootsma H, Nakamura T, Giacomelli R, Devauchelle-Pensec V, Knopf A, Bombardieri M, Trevisani V F, Hammenfors D, Pasoto S G, Retamozo S, Gheita T A, Atzeni F, Morel J, Vollenveider C, Horvath I F, Sivils K L, Olsson P, De Vita S, Sánchez-Guerrero J, Kilic L, Wahren-Herlenius M, Mariette X, Ramos-Casals M; Sjögren Big Data Consortium. How immunological profile drives clinical phenotype of primary Sjögren's syndrome at diagnosis: analysis of 10,500 patients (Sjögren Big Data Project). Clin Exp Rheumatol. 2018 May-June; 36 Suppl 112(3):102-112. Epub 2018 Aug. 14. PMID: 30156539.
10: Baldini C, Mosca M, Della Rossa A, Pepe P, Notarstefano C, Ferro F, Luciano N, Talarico R, Tani C, Tavoni A G, Bombardieri S. Overlap of ACA-positive systemic sclerosis and Sjögren's syndrome: a distinct clinical entity with mild organ involvement but at high risk of lymphoma. Clin Exp Rheumatol. 2013 March-April; 31(2):272-80. Epub 2013 Jan. 18. PMID: 23343785.
11: Kessel A, Toubi E, Rozenbaum M, Zisman D, Sabo E, Rosner I. Sjögren's syndrome in the community: can serology replace salivary gland biopsy?Rheumatol Int. 2006 February; 26(4):337-9. doi: 10.1007/s00296-005-0596-8. Epub 2005 Feb. 10. PMID: 15703951.
12: Pisoni C N, Reina S, Arakaki D, Eimon A, Carrizo C, Borda E. Elevated IL-1β levels in anti-Ro/SSA connective tissue diseases patients with prolonged corrected QTc interval. Clin Exp Rheumatol. 2015 September-October; 33(5):715-20. Epub 2015 Aug. 27. PMID: 26314292.
13: Lee I H, Kim S K, Kim M K. Dual anti-neutrophil cytoplasmic antibody-related pauci-immune crescentic glomerulonephritis in a patient with Sjögren's syndrome. Rheumatol Int. 2016 September; 36(9):1327-34. doi: 10.1007/s00296-016-3520-5. Epub 2016 Jul. 6. PMID: 27384449.
14: Yavuz S, Toker E, Bicakcigil M, Mumcu G, Cakir S. Comparative analysis of autoantibodies against a-fodrin in serum, tear fluid, and saliva from patients with Sjögren's syndrome. J Rheumatol. 2006 July; 33(7):1289-92. PMID: 16821267.
15: Turkçapar N, Olmez U, Tutkak H, Duman M. The importance of alpha-fodrin antibodies in the diagnosis of Sjögren's syndrome. Rheumatol Int. 2006 February; 26(4):354-9. doi: 10.1007/s00296-005-0607-9. Epub 2005 Jul. 1. PMID: 15990993.
16: Ng A L K, Choy B N K, Chan T C Y, Wong I Y H, Lai J S M, Mok M Y. Comparison of Tear Osmolarity in Rheumatoid Arthritis Patients With and Without Secondary Sjögren Syndrome. Cornea. 2017 July; 36(7):805-809. doi: 10.1097/ICO.0000000000001227. PMID: 28486313.
17: Pasoto S G, Natalino R R, Chakkour H P, Viana Vdos S, Bueno C, Leon E P, Vendramini M B, Neto M L, Bonfa E. EBV reactivation serological profile in primary Sjögren's syndrome: an underlying trigger of active articular involvement?Rheumatol Int. 2013 May; 33(5):1149-57. doi: 10.1007/s00296-012-2504-3. Epub 2012 Sep. 6. PMID: 22955798.
18: Altunoğlu A, Erten S̨, S̨ahin A, Is̨ιkoğlu S, Nes̨elioğlu S, Erzurum C, Erel Ö. Serum cystatin C is not an appropriate marker for kidney involvement in patients with primary Sjögren's syndrome. Int J Rheum Dis. 2017 March; 20(3):371-375. doi:10.1111/1756-185X.12513. Epub 2014 Nov. 4. PMID: 25367815.
19: Lim S A, Nam S, Kwok S K, Park S H, Chung S H. Serologic Markers Are Associated With Ocular Staining Score in Primary Sjögren Syndrome. Cornea. 2015 November; 34(11):1466-70. doi: 10.1097/ICO.0000000000000612. PMID: 26356755.
20: Ong S G, Choy C H. Autoimmune thyroid disease in a cohort of Malaysian SLE patients: frequency, clinical and immunological associations. Lupus. 2016 January; 25(1):67-74. doi: 10.1177/0961203315593164. Epub 2015 Jun. 25. PMID: 26113361.

1. A method for detecting anti-Ro antibody negative Sjögren's syndrome/Sjögren's disease without performing a lip biopsy comprising:
obtaining a biological sample from a patient suspected of having an anti-Ro antibody negative Sjögren's syndrome/Sjögren's disease; and
detecting if the biological sample has autoantibodies to at least one of: Geminin DNA Replication Inhibitor (GMNN), Kelch Domain Containing 8A (KLHDC8A), Microtubule Associated Protein RP/EB Family Member 1 (MAPRE1), Nucleoporin 50 (NUP50), or SKI Like Proto-Oncogene (SKIL).

2. The method of claim 1, further comprising the steps of detecting if the biological sample has autoantibodies to at least one of SSB or SKIL, and determining that the patient has Sjögren's syndrome/Sjögren's disease without performing a lip biopsy.

3. The method of claim 1, further comprising the step of detecting if the biological sample has autoantibodies to at least one additional biomarkers selected from ADP ribosylation factor GTPase activating protein 1 (ARFGAP1), Chromosome 9 Open Reading Frame 78 (C9orf78), Chromobox 3 (CBX3), Damage Specific DNA Binding Protein 1 (DDB1), GRB2 Associated Binding Protein 1 (GAB1), Heterogeneous Nuclear Ribonucleoprotein A/B (HNRNPAB), ISG15 Ubiquitin Like Modifier (ISG15), Multiple Coagulation Factor Deficiency 2 (MCFD2), NFU1 iron-sulfur cluster scaffold (NFU1), Pleckstrin Homology Domain Containing A4 (PLEKHA4), PML Nuclear Body Scaffold (PML), RNA Polymerase III Subunit H (POLR3H), POU Class 6 Homeobox 1 (POU6F1), RCAN Family Member 3 (RCAN3), SEC23 Interacting Protein (SEC23IP), SRY-box Transcription Factor 5 (SOX5), Small RNA Binding Exonuclease Protection Factor La (SSB), T-complex 10-like 3, pseudogene (TCP10L3), TPD52 Like 1 (TPD52L1), Triosephosphatase 1 (TPI1), Tripartite Motif Containing 21 (TRIM21), Ro60, Y RNA Binding Protein (TROVE2), Zinc Finger And BTB Domain Containing 46 (ZBTB46), and Zinc Finger Protein 655 (ZNF655).

4. The method of claim 1, wherein the autoantibodies are detected using an assay selected from at least one of: multiplex bead-based assay, capillary Western Blot, ELISA, flow cytometry, fluorimetry, microscopy, immunofluorescence, radioimmunoassay, immunoenzymatic assay, fluorescence activated cell sorting (FACS), differential display, representational difference analysis, microarray, Western blotting, immunohistochemical staining, immunocytochemical staining, dot blots, or surface plasmon resonance detection.

5. The method of claim 1, wherein the liquid biological sample is selected from a saliva, a blood, a plasma, a serum, or a tear sample.

6. The method of claim 1, further comprising the step of treating the patient negative for anti-Ro autoantibodies with a therapy that treats or reduces the symptoms of Sjögren's syndrome/Sjögren's disease.

7. An assay for detecting autoantibodies in anti-Ro antibody negative Sjögren's syndrome/Sjögren's disease comprising:
obtaining a biological sample from a patient suspected of having an anti-Ro antibody negative Sjögren's disease; and
detecting if the biological sample has autoantibodies to 1, 2, 3, 4, or 5, proteins selected from: GMNN, KLHDC8A, MAPRE1, NUP50, or SKIL, without performing a lip biopsy.

8. The assay of claim 7, further comprising the steps of detecting if the biological sample has autoantibodies to at least one of SSB or SKIL, and determining that the patient has SS without performing a lip biopsy.

9. The assay of claim 7, further comprising the step of detecting if the biological sample has autoantibodies to at least one additional biomarkers selected from ADP ribosylation factor GTPase activating protein 1 (ARFGAP1), Chromosome 9 Open Reading Frame 78 (C9orf78), Chromobox 3 (CBX3), Damage Specific DNA Binding Protein 1 (DDB1), GRB2 Associated Binding Protein 1 (GAB1), Heterogeneous Nuclear Ribonucleoprotein A/B (HNRNPAB), ISG15 Ubiquitin Like Modifier (ISG15), Multiple Coagulation Factor Deficiency 2 (MCFD2), NFU1 iron-sulfur cluster scaffold (NFU1), Pleckstrin Homology Domain Containing A4 (PLEKHA4), PML Nuclear Body Scaffold (PML), RNA Polymerase III Subunit H (POLR3H), POU Class 6 Homeobox 1 (POU6F1), RCAN Family Member 3 (RCAN3), SEC23 Interacting Protein (SEC23IP), SRY-box Transcription Factor 5 (SOX5), Small RNA Binding Exonuclease Protection Factor La (SSB), T-complex 10-like 3, pseudogene (TCP10L3), TPD52 Like 1 (TPD52L1), Triosephosphatase 1 (TPI1), Tripartite Motif Containing 21 (TRIM21), Ro60, Y RNA Binding Protein (TROVE2), Zinc Finger And BTB Domain Containing 46 (ZBTB46), and Zinc Finger Protein 655 (ZNF655).

10. The assay of claim 7, wherein the autoantibodies are detected using an assay selected from at least one of: capillary Western Blot, ELISA, flow cytometry, fluorimetry, microscopy, immunofluorescence, radioimmunoassay, immunoenzymatic assay, fluorescence activated cell sorting (FACS), differential display, representational difference analysis, microarray, Western blotting, immunohistochemical staining, immunocytochemical staining, dot blots, or surface plasmon resonance detection.

11. The assay of claim 7, wherein the liquid biological sample is selected from a saliva, a blood, a plasma, a serum, or a tear sample.

12. The assay of claim 7, further comprising the step of treating the patient negative for Ro autoantibodies with a therapy that treats or reduces the symptoms of SS.

13. A kit comprising a synthetic or recombinant polypeptide covalently attached to a solid support, wherein the synthetic or recombinant polypeptide selected from: GMNN, KLHDC8A, MAPRE1, NUP50, or SKIL.

14. The kit of claim 13, further comprising instructions for contacting the solid support with a biological sample from a patient suspected of having Sjögren's syndrome.

15. The kit of claim 13, wherein the solid support is selected from the group consisting of a multiwell plate, an enzyme-linked immunosorbent assay (ELISA) plate, a microarray, a bead, a porous strip, and a nitrocellulose filter.

16. The kit of claim 13, wherein the kit is an assay selected from the group consisting of a multiplex bead-based assay, capillary Western Blot, ELISA, flow cytometry, fluorimetry, microscopy, immunofluorescence, radioimmunoassay, immunoenzymatic assay, fluorescence activated cell sorting (FACS), differential display, representational difference analysis, microarray, Western blotting, immunohistochemical staining, immunocytochemical staining, dot blots, or surface plasmon resonance detection.

17. The kit of claim 13, further comprising a secondary antibody labeled directly or indirectly with a detectable moiety.

18. The kit of claim 13, further comprising the steps of detecting if the biological sample has autoantibodies to at least one of SSB or SKIL, and determining that the patient has SS without performing a lip biopsy.

19. The kit of claim 13, further comprising at least one additional biomarkers selected from ADP ribosylation factor GTPase activating protein 1 (ARFGAP1), Chromosome 9 Open Reading Frame 78 (C9orf78), Chromobox 3 (CBX3), Damage Specific DNA Binding Protein 1 (DDB1), GRB2 Associated Binding Protein 1 (GAB1), Heterogeneous Nuclear Ribonucleoprotein A/B (HNRNPAB), ISG15 Ubiquitin Like Modifier (ISG15), Multiple Coagulation Factor Deficiency 2 (MCFD2), NFU1 iron-sulfur cluster scaffold (NFU1), Pleckstrin Homology Domain Containing A4 (PLEKHA4), PML Nuclear Body Scaffold (PML), RNA Polymerase III Subunit H (POLR3H), POU Class 6 Homeobox 1 (POU6F1), RCAN Family Member 3 (RCAN3), SEC23 Interacting Protein (SEC23IP), SRY-box Transcription Factor 5 (SOX5), Small RNA Binding Exonuclease Protection Factor La (SSB), T-complex 10-like 3, pseudogene (TCP10L3), TPD52 Like 1 (TPD52L1), Triosephosphatase 1 (TPI1), Tripartite Motif Containing 21 (TRIM21), Ro60, Y RNA Binding Protein (TROVE2), Zinc Finger And BTB Domain Containing 46 (ZBTB46), Zinc Finger Protein 655 (ZNF655).

20. The method of claim 1, further:
obtaining a liquid biological sample from the patient suspected of having SS;
determining, by a computer device, that the patient is negative for Ro autoantibodies; and
detecting, by a computer device, if the biological sample has autoantibodies to 1, 2, 3, 4, or 5, proteins selected from: GMNN, KLHDC8A, MAPRE1, NUP50, or SKIL.

21. (canceled)

22. (canceled)

23. (canceled)

24. (canceled)

25. (canceled)
